(19) United States
(12) Patent Application Publication
(10) Pub. No.: US 2009/0036845 A1
(43) Pub. Date: Feb. 5, 2009

(54) Device For Delivery Of Agents To And Through The Human Scalp

(76) Inventor: David A. Smith, Gates Mills, OH (US)

Correspondence Address: Crockett & Crockett, Suite 200, 26020 Acero, Mission Viejo, CA, 92691, US

(21) Appl. No.: 12/184,112

(22) Filed: Jul. 31, 2008

Related U.S. Application Data
(60) Provisional application No. 60/963,034, filed on Aug. 1, 2007.

(51) Int. Cl. A45D 34/04; A61M 35/00

(52) U.S. Cl. 604/289; 132/212

(57) ABSTRACT

A method and device for administering minoxidil and other topical solutions to the scalp. The method entails administering a minoxidil preparation through a manifold that fits over the scalp of a patient.

This application claims priority to Provisional Patent Application 60/963,034 filed Aug. 1, 2007.

### FIELD OF THE INVENTIONS

The inventions described below relate the field of hair regrowth and treatments for baldness.

### BACKGROUND OF THE INVENTIONS

Minoxidil has proven to be an effective treatment for baldness. Specifically, it can stop and reverse ongoing hair loss and stimulate hair growth in adult men and women who are losing hair due to male or female pattern baldness (androgenic alopecia). However, minoxidil is only effective if properly applied. Minoxidil is applied with an eye-dropper, followed by careful massage of the applied solution to distribute it over the scalp. This is a tedious process, given that the typical user must apply minoxidil through the hair, and avoid wasting the minoxidil by wetting the hair instead of the scalp. Minoxidil must be applied twice daily for effective treatment. The desired result of renewed hair growth is obtained after several weeks or months of use. The twice-daily regimen must be continued indefinitely to maintain the effect, and if discontinued, any hair re-grown will be lost.

Due to the tedious nature of the regimen, most patients do not comply with the regimen. Because they do not maintain the regimen, the drug does not work, and patients discontinue the regimen, thereby abandoning an otherwise beneficial treatment.

In our co-pending application Rabin, et al., Device For Delivery Of Agents To And Through The Human Scalp, U.S. application Ser. No. 11/694,615, filed Mar. 30, 2007, we disclosed devices and methods which provide for convenient application of minoxidil to the scalp, to enhance patient compliance and/or permit a less rigorous treatment regimen. The device comprises a hat with a distribution manifold adapted to distribute minoxidil about the scalp, an injection port for injecting minoxidil into the manifold, and a reservoir for taking up a dose of minoxidil and injecting it into the manifold. The manifold includes numerous outlet nozzles that protrude, when the cap is in place, from the interior of the hat toward the scalp of the patient, and preferably contact the scalp. The hat is used by the patient by injecting a minoxidil solution into the manifold and hence through the nozzles onto the scalp, and thereafter wearing the hat for a period of time suitable to promote absorption of the distributed minoxidil.

### SUMMARY

The devices and methods described below provide uniform and efficient distribution minoxidil or other hair regrowth preparation from the various nozzles of a manifold comprising a cap-like array of nozzles suitable for wearing on the head of a user. The nozzles are arranged on the substrate, and fluid flow channels connecting the nozzles to a central distribution chamber are arranged such that the pathway from the distribution chamber to each nozzle is of substantially the same length, and the fluid flow channels are all of substantially identical internal diameter, such that the fluid flow paths leading from the distribution chamber to each of the nozzles results in substantially similar head loss. The nozzles are formed of elastomer nipples, with an end-wall thickness of about 1 mm and opening formed by closed slits in the end wall. This construction impedes flow until pressure within the nozzle exceeds a nominal pressure, such as 2 to 4 psi, whereupon the fluid pressure forces the slit open to allow fluid to pass through the nozzle.

### BRIEF DESCRIPTION OF THE DRAWINGS

FIG. 1 is a bottom perspective view of the cap to be used for distribution of minoxidil to the scalp of a patient.

FIG. 2 is a bottom view of a manifold used with the cap of FIG. 1.

FIGS. 3 and 4 illustrate the nozzles of the manifold.

### DETAILED DESCRIPTION OF THE INVENTIONS

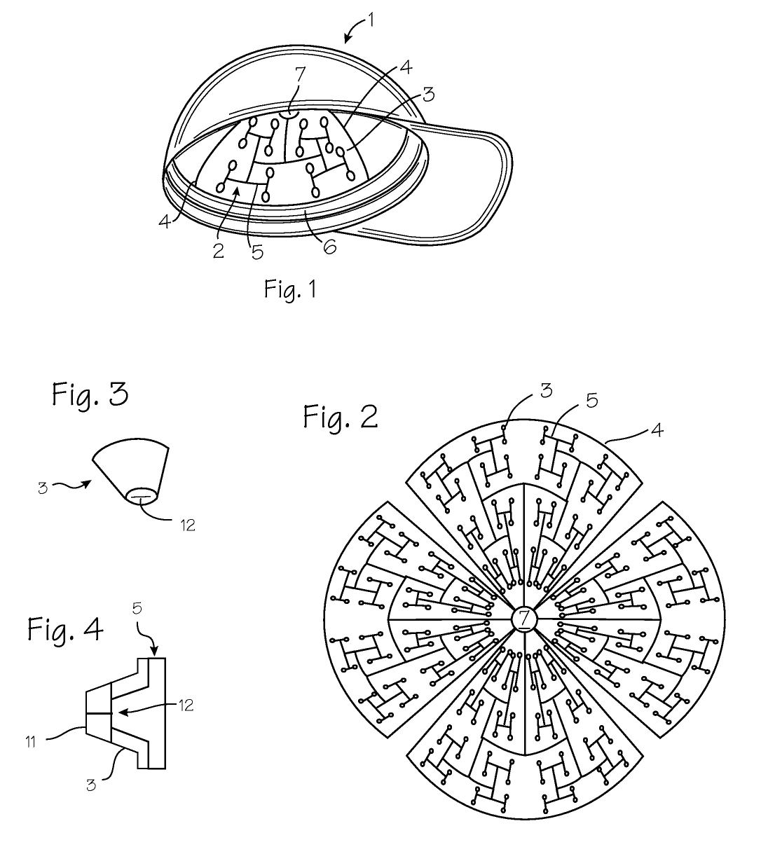

FIG. 1 is a bottom view of cap 1 to be used for distribution of hair-regrowth topical solutions, such as minoxidil preparations, to the scalp of a patient. The cap covers a manifold 2 supporting an array of nozzles 3 distributed about the inner surface of the hat. The manifold comprises several segments 4 with lumens 5 communicating with the nozzles. An occlusive band 6 is disposed about the perimeter of the cap, on the interior surface. The occlusive band may be elastic and tight-fitting to provide a tight-fitting sealing gasket between the user's scalp and the cap, or may be somewhat loose and merely promote creation of a closed environment of relatively high humidity immediately proximate the scalp.

The manifold 2 is shown more clearly in FIG. 2, which shows the arrangement of lumens within the manifold lumens and nozzles at the terminus of each fluid pathway of the manifold. The lumens 5 (shown in phantom) extend from the plenum or distribution chamber 7 in the center of the manifold, and are in fluid communication with the valved fitting and the several nozzles 3 providing outlet ports from the lumens. The manifold is preferably formed by molding a top sheet and a bottom sheet, with nozzles formed integrally on the bottom sheet and the lumens formed as channels in the bottom sheet, top sheet or both, to establish lumens when the top sheet and bottom sheet are secured together. As illustrated, the manifold is formed in several somewhat triangular segments 4, joined or formed together along segment edges so as to form a cap-like, roughly hemispherical manifold suitable for application to the head of a typical user. The manifold may also be formed of discrete tubing embedded in or covered with material such as neoprene, cloth, polyurethane or other suitable material, such that any tubing establishing the lumens, or manifold arms, are conveniently held in the desired array. This material, or the material of the cap, is provided in sufficient density to serve as a moisture barrier to enclose the environment immediately about the hair and/or scalp while in use.

The nozzles 3 are arranged on the interior surface of the cap so that each nozzle is fed by a channel of the same length. The channel pathway to each nozzle is established through one or more branch channels branching from a parent stem, such that for each flow path feeding a each nozzle, there are an identical number of branches, with an identical combined path length, feeding each nozzle. Thus, the channels feeding those nozzles nearest the distribution chamber are circuitous, running radially away from the distribution chamber and passing the nozzles to a point midway between those nozzles and the nozzles farthest from the distribution chamber, and splitting into two channels running in opposition directions circumferentially (relative to the circle or sphere defined by the several segments of the manifold) to midpoint between the nozzles, and thereafter splitting into additional channels running first radially up and down, and then circumferentially left and right, until the nozzles are reached. For the 64 nipple array illustrated, each nozzle is fed through a pathway comprising 6 segments. The segments are illustrated as rectilinear segments, but may be arcuate and smoothly curving. Additional arrays or portions of arrays of nozzles may be added to the cap, and these may be supplied via flow paths and channels in which the number of branches and/or combined path length are substantially different, such that the flow resistance in the pathway those nozzles in substantially different than the flow resistance in manifold as described herein, to provide for substantially more or less minoxidil to particular areas of the head.

The nozzles 3 are arranged on the interior surface of the cap so that, when the cap is worn, the nozzles will be disposed proximate the appropriate treatment target zones on the scalp, such as the vertex, occiput (back of the head), and frontal areas, and project toward the scalp from the interior surface of the manifold. Additional projections can be provided on the interior surface of the cap. Both the nozzles and the additional projections can be formed in various sizes and profiles, such as blunt flat distal faces or sharp or abrasive distal surfaces, to aid in distribution of the applied minoxidil, or to provide complimentary treatment such as micro-abrasion. Some of the projections may be taller than nearby nozzles to maintain a small gap between the nozzles and the scalp. The manifold and manifold arms are sized and dimensioned, and positioned within the cap, so that they lie over the desired portions of the scalp (vertex, occiput, and frontal areas) when the cap is worn in typical fashion (bill forward, hat band level on the head). The lumens of the manifold are sized to permit slow drainage of the minoxidil preparation, which may be watery or slightly viscous, and the lumens may be varied in size to suit the actual viscosity of the preparation used (commercially available minoxidil preparations such as Rogaine® have low viscosity, like water, though they are mixture of ethanol and slightly viscous propylene glycol).

FIGS. 3 and 4 illustrate the nozzles of the manifold. The nozzles 3 are formed of elastomeric nipples, with an end-wall 11 with a thickness of about 1 mm and opening formed by dimensionless slits 12 in the end wall. Absent a threshold pressure, the slits remain closed. This construction impedes flow until pressure within the nozzle exceeds a nominal pressure, such as 2 to 4 psi, whereupon the fluid pressure forces the slit open to allow fluid to pass through the nozzle. This facilitates even distribution of the minoxidil preparation and also prevents drippage of any minoxidil preparation retained within the manifold.

To use the cap, a user wears the cap in regular sessions, according to the appropriate treatment regimen, applying the minoxidil preparation through the cap for each session. Twice daily sessions, in which the cap is worn for about an hour, are adequate to provide effective treatment in lieu of the twice-daily eye-dropper application. A 1 cc volume of standard commercially available minoxidil preparation, or other amount effective to promote hair growth in other formulations, may be used. The injection of minoxidil may be accomplished before or after the user dons the cap, but the flushing of air or other fluid to clear the manifold should be done when the cap is in place. While wearing the cap, the user may engage in any activity, preferably for about 10 to 60 minutes, and more preferably for about 15 minutes. The user thereby holds the vapor barrier over the scalp for a period after application effective to enhance absorption of the minoxidil preparation by the scalp. After each session, the user may wash and style his hair as usual.

The device and method may be used to for hair re-growth using minoxidil or other hair re-growth compounds to re-grow hair, or prophylactically to prevent hair loss, or both. While minoxidil is generally referred to as a hair regrowth preparation, it also functions as a prophylactic hair maintenance preparation. Other compounds that perform solely as prophylactic hair maintenance preparations may be used in the method.

As mentioned above, the reservoir may be provided as a single dose reservoir, in either disposable or re-usable form. The re-usable reservoir is used by first connecting the suction end of the bellows/bladder pump to a larger reservoir of minoxidil preparation and operating the pump to fill the reservoir, and thereafter connecting the outlet end of the pump to the inlet port of the cap and operating the pump to pump the minoxidil preparation into the manifold, and then pump air into the manifold to flush any minoxidil preparation in the manifold onto the scalp. For minoxidil foam, an applicator that includes an active-treatment foam bolus followed by an inactive foam bolus equal in volume to the applicator dead volume may be used. A foam generator that mixes active agent in the first bolus of foam (10 cc, for example) and then cuts off the active agent for the next 7 cc of foam will provide the desired clearance effect without wasting active agent. Thus, the foam necessary to push a full dose of minoxidil through the nozzles will not be loaded with minoxidil.

The devices and methods described above have been described in relationship to the application of minoxidil, which is the currently predominant hair re-growth preparation available. Nonetheless, the devices and methods may be employed with any other hair re-growth compound or preparation, any hair loss prophylactic preparation, and with other cosmetic and/or therapeutic agents such as topical finasteride, minoxidil, ketoconazole, steroids, other anti-microbials, steroids, copper peptides for post-hair transplantation wound healing, anti-androgens, antimicrobials, spironolactone, spironolactone-like compounds, progesterone derivatives, betametazone valerate, ketoconazole, zinc salts, Zinc Pyrithione ZnP, finasteride, flutamide, dutasteride, melatonin, photo-activated compounds, lice treatments, cosmetic preparations such as scalp dye, hair dye, hair gel, conditioner, moisturizer, scalp oils, hair “volumizers,” vitamins, minerals, herbals, therapeutic water, zinc, iron, biotin, folic acid, anti-androgens, tretinoin, azelaic acid, and saw palmetto. The preparations may be provided in liquids of various viscosity, or in foams or other fluids, slurries or suspensions. Thus, while the preferred embodiments of the devices and methods have been described in reference to the environment in which they were developed, they are merely illustrative of the principles of the inventions. Other embodiments and configurations may be devised without departing from the spirit of the inventions and the scope of the appended claims.

I claim:

1. A device for applying a hair-regrowth topical solution to the scalp of a user, said device comprising:
a manifold sized and dimensioned to cover a portion of the scalp, said manifold having a interior surface adapted to appose the scalp and an outer surface, and a plurality of nozzles depending from the interior surface of the manifold, a fluid inlet port, and a plurality of channels establishing fluid communication between the fluid inlet port and the nozzles;
wherein the channel path length to each nozzle is established through one or more branch channels branching from a parent stem, such that for each branch feeding a particular nozzle, there are a substantially identical number of branches, with a substantially identical combined path length, feeding each nozzle;
said manifold shaped to fit over the scalp of the user.

2. An apparatus for applying topical solutions to the scalp of a user comprising:
a manifold sized to cover a portion of the user's scalp, the manifold having an interior surface adapted to appose the scalp and an outer surface, and a fluid inlet port;
a plurality of nozzles depending from the interior surface of the manifold;
a plurality of channels forming fluid paths in fluid communication between the fluid inlet port and the nozzles, each fluid path including one or more branch channels branching from a parent stem and having a substantially identical number of branches and a substantially identical combined path length.
